(19) United States
(12) Patent Application Publication
(10) Pub. No.: US 2023/0347167 A1
(43) Pub. Date: Nov. 2, 2023

(54) Lightweight smart hair-growing instrument with external power supply

(71) Applicant: Shenzhen Lescolton Electrical Appliance Co., Ltd., Shenzhen (CN)

(72) Inventor: Jianwen Liu, Shenzhen (CN)

(21) Appl. No.: 17/834,090

(22) Filed: Jun. 7, 2022

(30) Foreign Application Priority Data
Apr. 28, 2022 (CN) 202210470558.0

(51) Int. Cl. A61N 5/06; A61N 5/067

(52) U.S. Cl. CPC A61N 5/0617; A61N 5/067; A61N 2005/0626; A61N 2005/0647; A61N 2005/0642

(57) ABSTRACT

A lightweight smart hair-growing instrument with external power supply, which includes an headgear mechanism, light sources, a control mechanism, and a power source; the headgear mechanism is provided with a cavity for containing a user's head; the light sources are disposed in the cavity and faced to a centre of the cavity; the control mechanism are located at an outside of the headgear mechanism and electrically connected with the light sources; the power source is disposed in the control mechanism; the control mechanism and the light sources are connected electrically by a transmission line or driving connected by a wireless protocol signal; the power supply is integrated on the control mechanism independent with the headgear mechanism, which greatly reduces the whole heavy of the hair-growing instrument.

### FIELD OF THE INVENTION

The present invention relates to beauty device technology field, particularly, relates to a lightweight smart hair-growing instrument with external power supply.

### BACKGROUND OF THE INVENTION

A core technology of a laser hair-growing instrument is LLLT (low intensity laser therapy), a red light with 655 nm wavelength is used to activate vitality of hair follicles, accelerate blood circulation in a scalp and nutrition absorption of the hair follicle, reduce DHT (dihydro) on the hair follicles, promote ATP (adenosine triphosphate) as cellular energy to normally release and promote hair-growing. It also can regulate oil secretion, relieve the scalp tension, such as with drugs and nutrient solution can help nutrients and drugs better absorption.

Multiple medical organizations, such as University of Miami Miller School of Medicine, Department of Dermatology in University of Minnesota, Department of Dermatology, Clevel and Clinic, has developed clinical research about LLLT on hair-growing. Patients with alopecia between 25 and 60 years old underwent with 26 weeks of LLLT, the results demonstrated that the total response rate reached 93% for men and 90% for women.

Most traditional hair-growing instrument have the following structures: including a helmet and light sources. The helmet is worn on a human head, laser-ray output directions of the light sources are aligned to a scalp of the human head. After start up, the light sources output low-energy lasers to a preset position of the human head, thereby achieving the stimulating hair-growing effect. However, an electric storage unit of the traditional hair-growing instrument is disposed directly on itself. When the user uses the hair-growing instrument, the heavy of the electric storage unit and that of the hair-growing instrument are loaded directly on the user's head with, resulting in the the user's head bear too much heavy, which will damage the user's cervical spine.

### SUMMARY OF THE INVENTION

The object the present invention is to provide a lightweight smart hair-growing instrument with external power supply, aiming to resolve the technical problems that the electric storage unit of the traditional hair-growing instrument is disposed directly on itself, the heavy of the electric storage unit and that of the hair-growing instrument are loaded directly on the user's head with, resulting in the user's head bear too much heavy, which will damage the user's cervical spine.

In order to achieve the above object, the embodiment according to the present invention provides a lightweight smart hair-growing instrument with external power supply, which includes an headgear mechanism, light sources, a control mechanism, and a power source. The headgear mechanism is provided with a cavity for containing a user's head. The light sources are disposed in the cavity and faced to a centre of the cavity. The control mechanism is located at an outside of the headgear mechanism and electrically connected with the light sources. The power source is disposed in the control mechanism. The control mechanism and the light sources are connected electrically by a transmission line or driving connection through a wireless protocol signal.

Preferably, the headgear mechanism includes a base and a frame. The cavity is formed on a bottom surface of the base, the frame is detachably connected with a top surface of the base away from the cavity. The light sources are disposed between the base and the frame. The top surface of the base is provided with a conversion unit connected with the control mechanism and connected electrically with the light sources.

Preferably, the control mechanism and the conversion unit are connected electrically through a USB connecting line and a TYPE-C connecting line.

Preferably, the control mechanism is provided with a transmitting unit. The conversion unit includes a receiving antenna and a driving module. The receiving antenna and the transmitting unit are signal connection, and the driving module is connected electrically to the light sources.

Preferably, the frame includes a shell and a few of positioning columns. The shell is provided with an accommodating cavity for covering the base. The positioning columns are evenly distributed on an inner wall of the shell. Each of the positioning columns is provided with a screw hole, and the base is threaded to the positioning columns by a screws.

Preferably, each of the positioning columns includes a connecting rod and a strengthening assembly. One end of the connecting rod is fixed to the inner wall of the shell, the other end of the connecting rod extends towards the base. The strengthening assembly is disposed between a side wall of the connecting rod and the inner wall of the shell. The screw hole is formed at one end of the connecting rod away from the shell. The strengthening assembly is used for improving the structural stability of the connecting rod.

Preferably, the inner wall of the base is provided with at least one massage assembly. All the massage assemblies are distributed evenly around a center of the user's head.

Preferably, the number of the massage assemblies is four, which are distributed evenly on the bottom surface of the base.

Preferably, each of the massage assemblies includes a clamping portion, a connecting portion, and a multiple of columns. The bottom surface of the base is provided with a few of mounting slots, the clamping portion is clamped with one of the mounting slots. The connecting portion is fixed on an end, away from the mounting slots, of the clamping portion and extends into the cavity. The multiple of columns are distributed evenly on an end, facing to the cavity, of the connecting portion.

Preferably, the lengths of the columns gradually decrease from the edge of the connecting portion to the center of the connecting portion, thereby forming a groove.

One or more of the above technical schemes in the lightweight smart hair-growing instrument with external power supply provided in the embodiment in the invention have at least one of the following technical effects:

When the hair-growing instrument is using, the control mechanism containing the power supply drives directly the light sources independent from the control mechanism and contained in the headgear mechanism to work. The light sources emit light to illuminate a user's scalp, thereby stimulating the hair-growing. In the work process of the hair-growing instrument, the power supply is always in the control mechanism independent from the headgear mechanism, the heavy of the power supply is not bore by the user. Compared with the traditional hair-growing instrument that the power supply is disposed directly on it, the heavy of the power supply is bore by the user's head, resulting in the user's head bearing too much heavy, which will damage the user's cervical spine. In the embodiment, the lightweight smart hair-growing instrument with external power supply, the power supply is integrated on the control mechanism independent with the headgear mechanism, which greatly reduces the whole heavy of the hair-growing instrument.

### BRIEF DESCRIPTION OF THE DRAWINGS

In order to explain more clearly the technical scheme in the embodiments of the invention, a brief introduction is given below to the attached drawings needed in the embodiments or the prior art description. Obviously, the attached drawings in the following description are only some embodiments of the invention. For ordinary technicians in this field, other drawings can be obtained according to these drawings without paying creative labor.

FIG. 1 is a perspective view of the lightweight smart hair-growing instrument with external power supply according to the invention.

FIG. 2 is a section view of the control mechanism according to the invention.

FIG. 3 is a circuit construction block of the lightweight smart hair-growing instrument with external power supply according to the invention.

FIG. 4 is an exploded view of the lightweight smart hair-growing instrument with external power supply according to the invention.

FIG. 5 is a section view of the control mechanism adopting the wireless remote according to the invention.

FIG. 6 is a circuit construction block of the lightweight smart hair-growing instrument with external power supply adopting the wireless remote according to the invention.

FIG. 7 is a perspective view of the frame according to the invention.

FIG. 8 is a bottom view of the frame showing in FIG. 7.

FIG. 9 is an enlarge view of the B part in FIG. 7.

FIG. 10 is a perspective view of the massage mechanism according to the invention.

FIG. 11 is the other perspective view of the massage mechanism according to the invention.

FIG. 12 is an enlarge view of the A part in FIG. 1.

FIG. 13 is a perspective view of the base according to the invention.

FIG. 14 is an enlarge view of the C part in FIG. 13.

### DETAILED DESCRIPTION OF ILLUSTRATED EMBODIMENTS

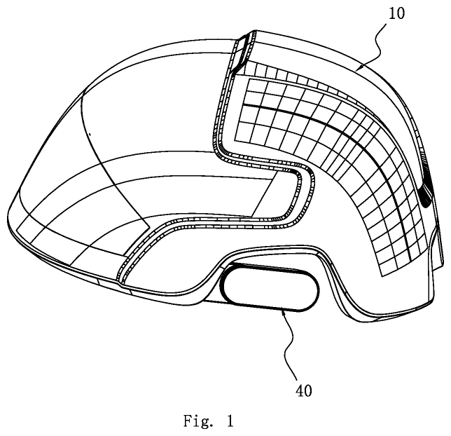

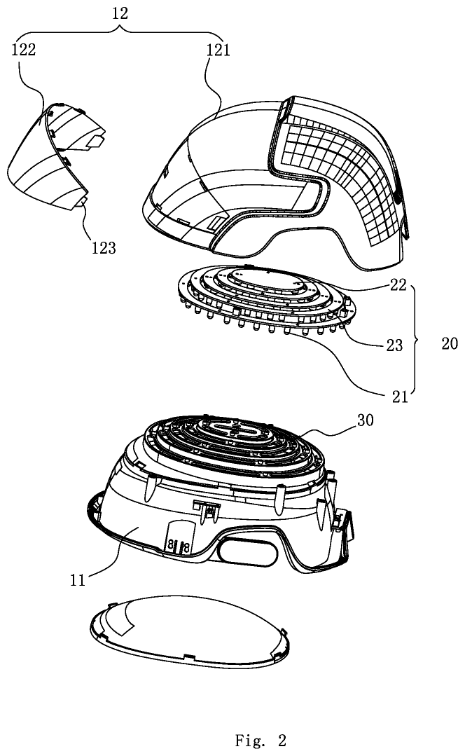

The embodiments of the present invention are described in details below, and the embodiments are shown in the attached figures, where the same or similar label from beginning to end represents the same or similar element or element with the same or similar function. The embodiments described below by reference to FIGS. 1-6 are exemplary and are intended to be used to explain embodiments of the present invention and can not be understood as limitations to the present invention.

The embodiments of the present invention are described in details below, and the embodiments are shown in the attached figures, where the same or similar label from beginning to end represents the same or similar element or element with the same or similar function. The embodiments described below by reference to FIGS. 1-6 are exemplary and are intended to be used to explain embodiments of the present invention and can not be understood as limitations to the present invention.

In the description of the embodiment of the present invention, it needs to be understood that the azimuth or position relation indicated in the terms “length”, “width”, “upper”, “lower”, “front”, “rear”, “left”, “right”, “vertical”, “horizontal”, “top”, “bottom” “inside”, “outside” and so on are based on the azimuth or position relation shown in the attached drawing, only for the convenience of describing the embodiment of the present invention and simplifying the description, rather than indicating or implying that the device or element must have a specific orientation, with a specific orientation and operation, so it can not be understood as a limitation to the present invention.

Furthermore, the terms “first”, “second” are used only for descriptive purposes and it can not be understood as indicating or implying relative importance or implicitly the number of technical features indicated. Thus, the qualification of “first” and “second” features may include one or more of them explicitly or implicitly. In the description of the embodiments of the invention, the meaning of “multiple” is two or more, unless otherwise specified.

In the embodiment of the invention, unless other specified and qualified, the terms “installation”, “connection”, “fixed” and other terms shall be broadly understood, for example, it may be fixed connection, detachable connection, or integration; it may be mechanical connection or electrical connection; it may be directly connected or indirectly connected by intermediate media, it may be internal connection of two components or the interaction of two components. For ordinary technicians in the field, the specific meaning of the above term in the embodiment of the invention can be understood according to the specific circumstances.

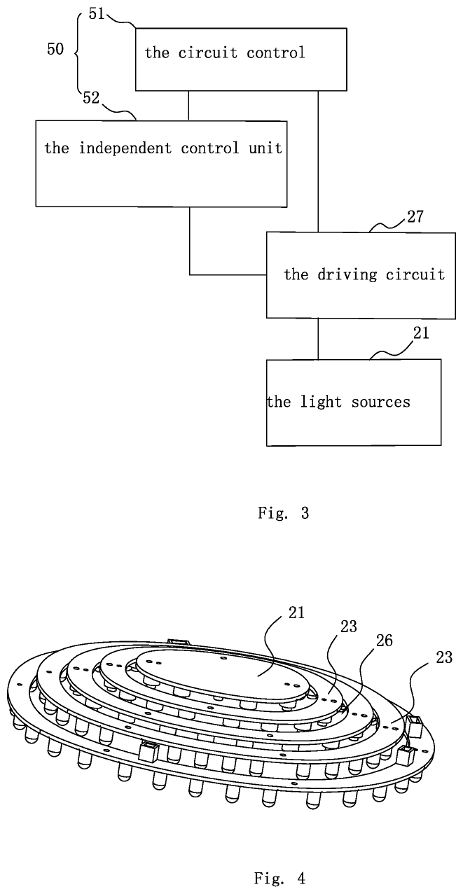

In an embodiment, referring to FIG. 3, a lightweight smart hair-growing instrument with external power supply includes an headgear mechanism 10, light sources 20, a control mechanism 30, and a power source 50. The headgear mechanism 10 is provided with a cavity 11 for containing the user's head. The light sources 20 are disposed in the cavity 11. The control mechanism 30 is located at the outside of the headgear mechanism 10 and electrically connected with the light sources 20. The power source 50 is disposed in the control mechanism 30. The control mechanism 30 and the light sources 20 are connected electrically by a transmission line or driving connection through a wireless protocol signal.

In the embodiment, the control mechanism 30 includes a shell 31 and a control circuit module 32. The shell 31 is provided with a mounting cavity 33 for installing the control circuit module 32. The shell is provided with a loading cavity 34 for containing the power supply 50. The shell 31 is provided with a button 35 for driving the control circuit module 32.

Specifically, when the hair-growing instrument is using, the control mechanism 30 containing the power supply 50 drives directly the light sources 20 independent from the control mechanism 30 and contained in the headgear mechanism 10 to work. The light sources 20 emit light to illuminate a user's scalp, thereby stimulating the hair-growing. In the work process of the hair-growing instrument, the power supply 50 is always in the control mechanism 30 independent from the headgear mechanism 10, the heavy of the power supply 50 is not bore by the user. Compared with the traditional hair-growing instrument that the power supply 50 is disposed directly on it, the heavy of the power supply 50 is bore by the user's head, resulting in the user's head bearing too much heavy, which will damage the user's cervical spine. In the embodiment, the lightweight smart hair-growing instrument with external power supply, the power supply 50 is integrated on the control mechanism 30 independent with the headgear mechanism 10, which greatly reduces the whole heavy of the hair-growing instrument.

Referring to FIG. 4, in another embodiment, the headgear mechanism 10 includes a base 12 and a frame 13. The cavity 13 is formed on a bottom surface 121 of the base 12, the frame 13 is detachably connected with a top surface 122 of the base 12. The light sources 20 are disposed between the base 12 and the frame 13. The top surface 122 of the base 12 is provided with a conversion unit 14 connected with the control mechanism 30 and connected electrically with the light sources 20. Specifically, the conversion unit 14 is used as an electric energy transfer and a transmission structure, which greatly reduced the heavy of the power supply 50, and effectively improve the lightness and the safety of the hair-growing instrument.

In another embodiment, the control mechanism 30 and the conversion unit 14 are connected electrically through a USB connecting line and a TYPE-C connecting line. It should be noticed that those skilled in the art should understood that any structure can realize the power transmission except the above USB connecting line and a TYPE-C connecting line should be in the protecting range of the present invention.

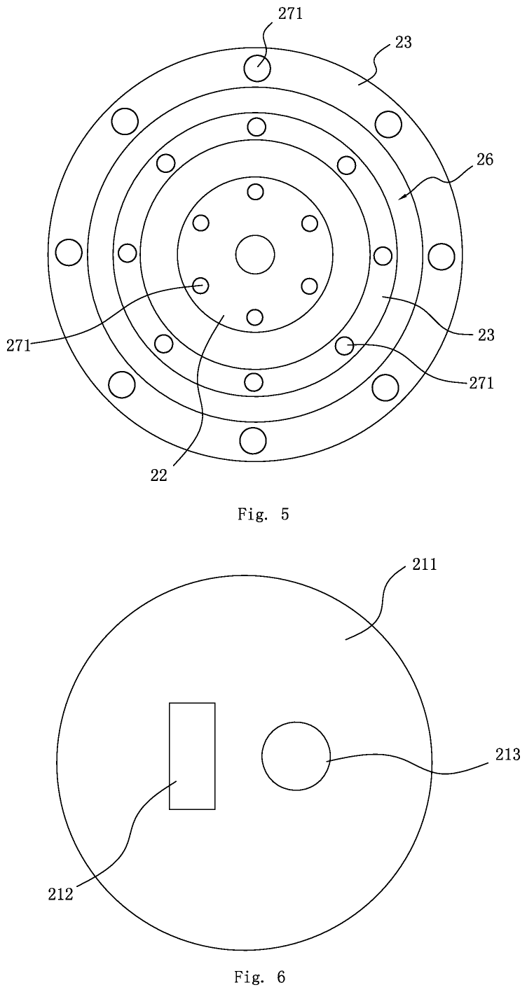

Referring to FIG. 5-6, in the embodiment, the control mechanism 30 is provided with a transmitting unit 36. The conversion unit 14 includes a receiving antenna 141 and a driving module 142. The receiving antenna 141 and the transmitting unit 36 are signal connection, and the driving module 142 is connected electrically to the light sources 20. Specifically, the transmitting unit 36 is connected electrically to the control circuit module 32. When the user pushes the button and sends signals to the control circuit module 32, the transmitting unit 36 sends the corresponding signal to the receiving antenna 141. After receiving the signals, the driving module 42 drives the light sources 20 to operate, thereby realizing remote control operation. The transmitting unit 36 and the receiving antenna 141 can be signal connection by a Bluetooth, a infrared, or NFC transmission protocol. Using remote control instead of line control is beneficial to prevent signal interference in the transmission line integrated control circuit and improve the control effect.

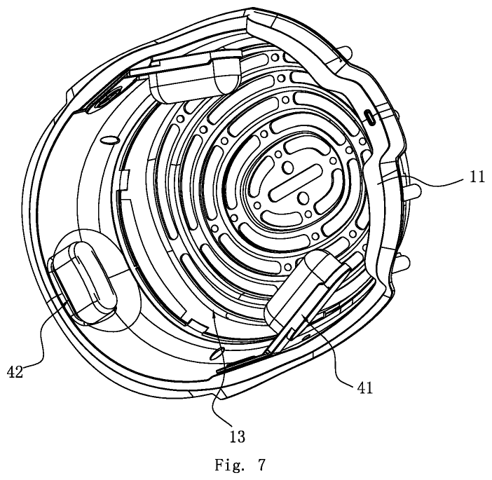

Referring to FIG. 7, in the other embodiment, the frame 13 includes a shell 131 and a few of positioning columns 132. The shell 131 is provided with an accommodating cavity 135 for covering the base 12. The positioning columns 132 are evenly distributed on an inner wall 131a of the shell 131. Each of the positioning columns 132 is provided with a screw hole 136, and the base 12 is threaded to the positioning columns 132 by screws 137. Specifically, the shell 131 covers the base 12 in a bowl-like structure. The positioning columns 132 are evenly distributed on both sides of the shell 131. The shell 131 and the positioning columns 132 are made of plastic material, formed by injection molding machine.

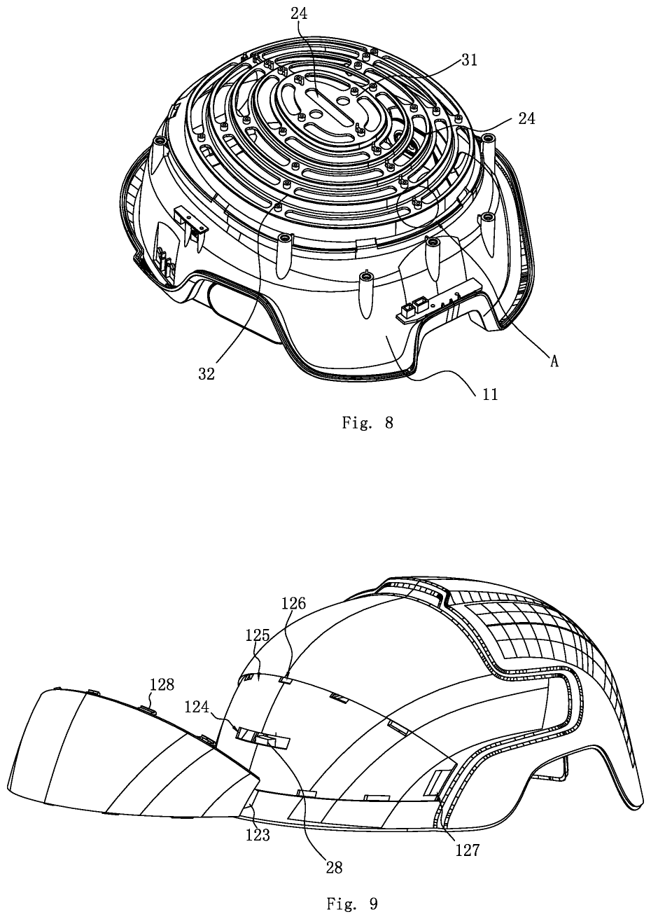

Referring to FIG. 8 and FIG. 9, in another embodiment, each of the positioning columns 132 includes a connecting rod 133 and a strengthening assembly 134. One end of the connecting rod 133 is fixed to the inner wall of the shell 131, the other end of the connecting rod 133 extends towards the base 12. The strengthening assembly 134 is disposed between the side wall of the connecting rod 133 and the inner wall of the shell 131. The screw hole 136 is formed at one end of the connecting rod 133 away from the shell 131. The strengthening assembly 134 is used for improving the structural stability of the connecting rod 133 to prevent the connecting rod 133 from shifting during repeatedly wearing, resulting the shell 131 separating, affecting the use.

Referring to FIG. 9, in another embodiment, the strengthening assembly 134 includes at least a reinforcing plate 139. One end of the reinforcing plate 139 is fixed to the inner wall of the accommodating cavity 135, the other end of the reinforcing plate 139 is fixed to the outer wall of the connecting rod 133. The reinforcing plate 139 is made of plastic, and the reinforcing plate 139 is integrated with the inner wall of the accommodating cavity 135. The reinforcing plate 139 is arranged in an arc shape near the inner wall edge of the accommodation cavity 135, and the reinforcing plate 139 forms the connecting rod 133 and the accommodation cavity 135 to further improve the stability of the frame 13.

Referring to FIG. 8, in another embodiment, the number of the reinforcing plate 139 is two. The two reinforcing plates 139 intersect vertically to each other, and the connecting rod 133 is located at the intersecting position of the two reinforcing plates 139. Specifically, the two reinforcing plates 139 fix the connecting rod 133 from two direction. Wherein, the intersection position E between the two reinforcing plates 139, the connecting position D between one of the reinforcing plates 139 and the inner wall of the accommodation cavity 135, and the connecting position C of the other of the reinforcing plates 139 and the inner wall of the accommodation cavity 135 forms a stable triangle support structure, which can improve the strength of the shell 131 without substantially reinforcing the thickness of the shell 131. The above structure not only satisfies the principle of product durability, but also can maintain the hair-growing instrument light, further improve the market reputation of products, which is conducive to the development of enterprises.

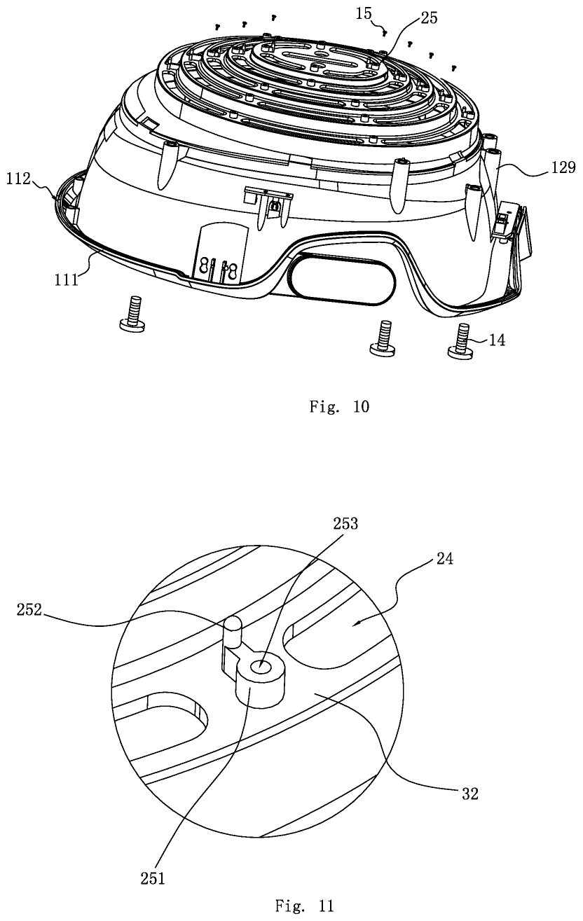

Referring to FIG. 4 and FIG. 10, in another embodiment, the bottom surface 121 of the base 12 is provided with at least one massage assembly 40. Preferably, the number of the massage assemblies 40 is four. All the massage assemblies 40 are distributed evenly on the bottom surface 121 of the base 12 around the center of the user's head. Each of the massage assemblies 40 contacts with the user's scalp so that there is always a gap between the user's scalp and the inner wall of the base 12, which prevent the user's scalp from burning because of too close to the light source. At the same time, the massage assemblies 40 can massage the user's scalp and improve the comfortable of the hair-growing instrument.

Referring to FIG. 12, in another embodiment, each of the massage assemblies 40 includes a clamping portion 411, a connecting portion 412, and a multiple of columns 413. The bottom surface of the base 12 is provided with a few of mounting slots 414, the clamping portion 411 is clamped with one of the mounting slots 414. The connecting portion 412 is fixed on an end, away from the mounting slots 414, of the clamping portion and extends into the cavity 11. The multiple of columns 413 are distributed evenly on an end, facing to the cavity 11, of the connecting portion 412.

Specifically, the opening edge of each of the mounting slots 414 is provided with a positioning ring 415. The positioning ring 415 is clamped with the connecting portion 412 when the clamping portion 411 is clamped with one of the mounting slots 414. Each of the massage assemblies 40 is disassembly connected with the base 12 so that the massage assemblies 40 can be changed according the demands of different people, thereby improving the practicality of the hair-growing instrument. The columns 413 is made of an elastic silicone.

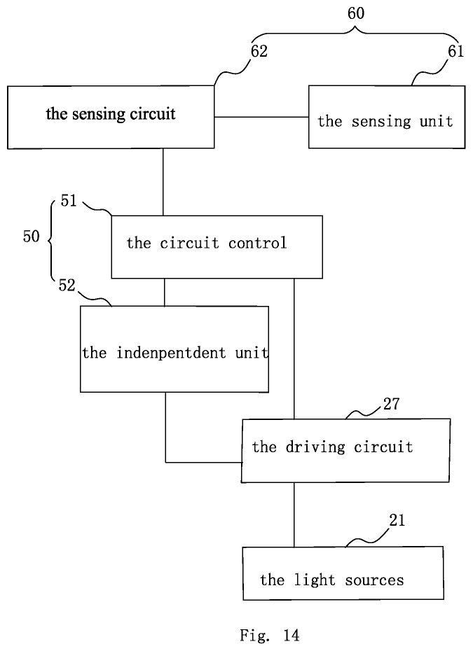

Referring to FIG. 14, in another embodiment, the lengths of the columns 413 gradually decrease from the edge of the connecting portion 412 to the center of the connecting portion 412, thereby forming a groove 414. Specifically, when a user wears the hair-growing instrument, the user's head contacts the columns 413 with larger lengths located on the edge of the connecting portion 412 and presses these columns 413 towards both sides, the user's head also contacts the columns 413 with less lengths located on the center of the connecting portion 412, but does not or less press the columns 413 with less lengths located on the inner side so that those columns 413 do not deform or less deform. In the traditional massage assemblies, all of the columns have the same lengths so that the user's head will press all of them to deform, resulting in that the traditional massage assemblies are not durable. The groove 414 formed by the columns 413 with different length can avoid the deformation of the columns 413 at the center of the connection portion 412, thereby extending the service life of the columns 413 to further improve the durability of the product.

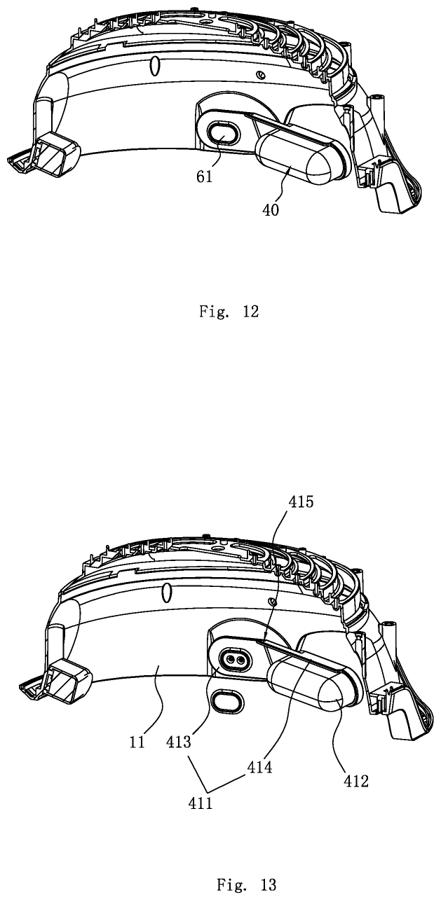

Referring to FIG. 13-14, in another embodiment, the edge of the base 12 folds to form a connecting edge 15, the end, away from the base 12, of the connecting edge 15 forms a positioning slot 151, the bottom surface of the frame 13 protrudes a convex-ring 138 clamped with the positioning slot 151. When preparing the headgear mechanism 10, the base 12 and the outer frame 13 are initially attached by the positioning groove 151 and the convex-ring 138, which is beneficial to improve the accuracy of the screws 137.

Referring to FIG. 14, in another embodiment, the connecting edge 15 is provided with holes for heat dissipation 152 communicated with the accommodation cavity 135. The number of heat dissipation holes 152 is multiple groups that are evenly spaced out along the length direction of the connecting edge 15. Specifically, when the users wears the hair-growing instrument, the connecting edge 15 contacts directly with the space between the user's temples and ears. If the hair-growing instrument is used for a long time, the connecting edge 15 will inevitably heat up, and which is easy to cause discomfort to the user. The heat dissipation holes 152 can expel the heat quickly, which will effectively reduce the temperature of the connecting edge 15 and improve the comfortable of the hair-growing instrument.

The above is only the preferred embodiment of the invention and is not intended to limit the invention. Any modification, equivalent replacement and improvement made within the spirit and principles of the invention shall be included in the protection scope of the invention.

What is claimed is:

1. A lightweight smart hair-growing instrument with external power supply, wherein comprising,
an headgear mechanism, having a cavity for containing a user's head;
light sources, arranged in said cavity;
a control mechanism, located at an outside of said headgear mechanism and electrically connected with said light sources;
a power source, arranged in said control mechanism; and,
said control mechanism and said light sources are connected electrically by a transmission line or driving connection through a wireless protocol signal.

2. The lightweight smart hair-growing instrument with external power supply according to claim 1, wherein said headgear mechanism includes a base and a frame; said cavity is formed on a bottom surface of said base, said frame is detachably connected with an end, away from said cavity, of said base; said light sources are disposed between said base and said frame; said top surface of said base is provided with a conversion unit connected with said control mechanism and connected electrically with said light sources.

3. The lightweight smart hair-growing instrument with external power supply according to claim 2, wherein said control mechanism and said conversion unit are connected electrically through a USB connecting line and a TYPE-C connecting line.

4. The lightweight smart hair-growing instrument with external power supply according to claim 2, wherein said control mechanism is provided with a transmitting unit; said conversion unit includes a receiving antenna and a driving module; said receiving antenna and said transmitting unit are signal connection, and said driving module is connected electrically to said light sources.

5. The lightweight smart hair-growing instrument with external power supply according to claim 2, wherein said frame includes a shell and a few of positioning columns; said shell is provided with an accommodating cavity for covering said base; said positioning columns are evenly distributed on an inner wall of said shell; each of said positioning columns is provided with a screw hole, and said base is threaded to said positioning columns by screws.

6. The lightweight smart hair-growing instrument with external power supply according to claim 5, wherein each of said positioning columns includes a connecting rod and a strengthening assembly; one end of said connecting rod is fixed to said inner wall of said shell, the other end of said connecting rod extends towards said base; said strengthening assembly is disposed between a side wall of said connecting rod and said inner wall of said shell; said screw hole is formed at one end of said connecting rod away from said shell.

7. The lightweight smart hair-growing instrument with external power supply according to claim 1, wherein said bottom surface of said base is provided with at least one massage assembly; all said massage assemblies are distributed evenly around a center of said user's head.

8. The lightweight smart hair-growing instrument with external power supply according to claim 7, wherein a number of said massage assemblies is four, which are distributed evenly on said bottom surface of said base.

9. The lightweight smart hair-growing instrument with external power supply according to claim 7, wherein each of said massage assemblies includes a clamping portion, a connecting portion, and a multiple of columns; said bottom surface of said base is provided with a few of mounting slots, said clamping portion is clamped with one of said mounting slot; said connecting portion is fixed on an end, away from said mounting slots, of said clamping portion and extends into said cavity; said multiple of columns are distributed evenly on an end, facing to said cavity, of said connecting portion.

10. The lightweight smart hair-growing instrument with external power supply according to claim 9, wherein lengths of said columns gradually decrease from an edge of said connecting portion to a center of said connecting portion, thereby forming a groove.
